(19) United States
(12) Patent Application Publication
(10) Pub. No.: US 2018/0035783 A1
(43) Pub. Date: Feb. 8, 2018

(54) COSMETIC PRODUCT COMPRISING A METAL CAN, AND THE CONTENTS OF SAME

(71) Applicant: BEIERSDORF AG, Hamburg (DE)

(72) Inventors: Anja BAUER, Hamburg (DE); Volker KALLMAYER, Hamburg (DE); Peter STEIDLE, Hamburg (DE); Klaus-Peter STANGE, Wentorf (DE)

(21) Appl. No.: 15/551,596

(22) PCT Filed: Feb. 8, 2016
(86) PCT No.: PCT/EP2016/052612

§ 371 Date: Aug. 17, 2017

(30) Foreign Application Priority Data
Feb. 27, 2015 (DE) 10 2015 203 565.4

(51) Int. Cl. A45D 40/00; A61K 8/37; A61K 8/891; B65D 43/02; A61K 8/36

(52) U.S. Cl. CPC A45D 40/0068; B65D 43/0231; A61K 8/361; A61K 8/891; A61K 8/375; A45D 2200/05; B65D 2543/00092; B65D 2543/00277

(57) ABSTRACT

Disclosed is a cosmetic product comprising a metal can which contains a cosmetic preparation and comprises a metal container (A) with thread (A) and a lid (D) having a thread (D) and consisting of the same metal as the container. The lid is designed such that it can be screwed onto or unscrewed from the container by a rotational movement. The cosmetic preparation comprises an emulsion of one or more hydrophilic phases and one or more lipophilic phases. The one or more lipophilic phases contain at least one lipid with a viscosity at 25° C. of less than 15 mPa·s and a spreading coefficient at 25° C. of at least 700 mm²/10 minutes.

The invention relates to a cosmetic product comprising a metal can composed of a container part with rotation thread (container thread), in the interior of which a cosmetic preparation is kept, and also a lid part made of metal with a thread (lid thread), wherein the lid part is unscrewable and screwable onto the container part in a closing manner by means of the lid thread and the container thread via rotational movement, i.e., designed to be openable and recloseable via a screw closure.

Cosmetic products comprising metal cans having contents, for example creams, fats and the like, have been used for at least a hundred years.

These known tin cans are disadvantageous in that either the choice of the wall material is limited, since screwing movement of the lid on the container part almost inevitably results in abrasion, which leads to unsightly signs of soiling, or containers without screw threads have to be chosen, which, however, can lead to leakage problems.

It is an object of the invention to provide a metal can having a profile edge which has a circular plane-surface and is especially suitable for sealing and, at the same time, does not exhibit the described disadvantages of the prior art.

These objects are achieved according to the invention by a cosmetic product comprising

a metal can comprising
aa) a metallic container part (A) with rotation thread (A′),
   aaa) in the interior of which a cosmetic preparation (Z) is present,
      and also
bb) a lid part (D) with a thread (D′) made of the same metal as the container part,
   wherein the lid part (D) is unscrewable and screwable onto the container part (A) in a closing manner by means of the lid thread (D′) and the container thread (A′) via rotational movement, i.e., designed to be openable and reclosable via screwing movement,
   characterized in that the cosmetic preparation comprises an emulsion of
cc) one or more hydrophilic phases
dd) one or more lipophilic phases,
   wherein
ee) in which the lipid phase contains at least one lipid having a viscosity of less than 15 mPa·s (at 25° C.), which lipid has a spreading value of at least 700 mm²/10 minutes (at 25° C.).

Spreading is the property of low-viscosity oils, which property is often desired, though also often undesired in other situations, and is predominantly based on capillary forces, to be particularly easily distributed as a thin layer on substrates or else on the skin. This may be advantageous in skincare. This property may have a disadvantageous effect in the packaging of such oils or preparations containing said oils. A measure of spreading capacity is the spreading coefficient, which, for example, assumes particularly high values in defoamers and foam inhibitors.

Barry and Grace developed a method for determining spreading behavior (J. Pharmac. Sci. 61, 335 [1972] and Beyer developed a model test system for testing spreading behavior (Arch. Pharm. [Weinh.] 310, 729 [1977]; Chem. Abstr. 88, No. 12-79017 [1978]). Beyer further reports on the model-based spreading of ointments in Arch. Pharm. 310, 473 and 858 (1977); Zbl. Pharm. 118, 51 (1979). Pascale et. al. report on the spreading capacity of various liquid excipients based on fats or fat-like substances (Cosmet. Toiletries 100, No. 10, 75 [1985]).

The measurement unit of the spreading coefficient is that of the quotient formed from the spreading surface, across which the spreading takes place, and the spreading time, within which the spreading takes place. It is usually specified in [mm²/10 minutes].

Within the context of the present disclosure, the expression “lipids” is occasionally used as an umbrella term for fats, oils, waxes and the like, as is thoroughly familiar to a person skilled in the art. The terms “oil phase” and “lipid phase” are also used synonymously.

Preferably, preparations according to the invention contain up to 35% by weight of a lipid phase.

Advantageously, the oils used according to the invention are selected from the group of substances listed in the following table:

TABLE 1

| | | | Spreadability |
|---|---|---|---|
| | | Viscosity, | (20 μl/red- |
| | Analogous | mPas | band filter |
| Trade name | INCI name | mPa · s | mm²/10 min |
| Solvent IH | Isohexadecane | 8 | 990 |
| | Isoeicosane | 12 | 800 |
| Cegesoft ® | Octyl | 11 | 910 |
| C24 | palmitates | | |
| | Isopropyl | 9 | 910 |
| | stearate | | |
| Estol ® 1540 | Octyl cocoate | 10 | 930 |
| EHC | | | |
| Finsolv ® TN | C₁₂₋₁₅ alkyl | 14 | 730 |
| | benzoates | | |
| Cetiol ® OE | Dicaprylyl | 8 | 1020 |
| | ether | | |
| DUB DNPG | Neopentyl | 13 | 830 |
| | glycol | | |
| | diheptanoate | | |
| Miglycol ® | Propylene | 12 | 855 |
| 840 | glycol | | |
| | dicaprylate/ | | |
| | dicaprate | | |
| DC Fluid 345 | Cyclomethicone | 5 | 770 |
| | Isopropyl | 7.1 | 1590 |
| | palmitate | | |
| Cetiol ® B | Dibutyl | 5.5 | 935 |
| | adipate | | |
| DUB VCI 10 | Isodecyl | 3.9 | 962 |
| | neopentanoate | | |
| Cetiol ® CC | Dicaprylyl | 7.26 | 875 |
| | carbonate | | |
| Cetiol ® S | Dioctyl | 14.41 | 723 |
| | cyclohexane | | |
| | Dihexyl | 3.8 | 1056 |
| | carbonate | | |
| | Dihexyl ether | 1.87 | 1174 |
| Ecolane ® 130 | Cycloparaffin | 5.22 | 908 |
| Softcutol ® O | Ethoxydiglycol | 13.07 | 804 |
| | oleates | | |
| Transcutol ® | Ethoxydiglycol | 4.14 | 999 |
| CG | | | |
| Dermofeel ® | Butylene | 12 | 800 |
| BGC | glycol | | |
| | caprylate/caprate | | |
| Prisorine ® | Octyl | 15 | 800 |
| 2036 | isostearate | | |
| Tegosoft ® SH | Stearyl | 13 | 755 |
| | heptanoate | | |
| Tegosoft ® DC | Decyl cocoate | 10.3 | 788 |
| Transcutol ® | Ethoxy | 4.27 | 962 |
| P | diglycol | | |
| Arlasolv ® | Dimethyl | 10 | 880 |
| DMI | isosorbide | | |

Such a metal can has a sufficiently broad, planar surface on the upper edge for sealing, meaning that overlays can be firmly sealed onto the can in a manner known per se. To this end, a flexible film composed of metal, plastic or their composite, for example, is sealed onto the filled can by means of heat sealing or ultrasound, the inner protective coating applied before the shaping of the can having thermoplastically sealable or ultrasound-weldable properties.

It is also possible to subsequently apply a suitable sealing material, such as high-melting-point wax, adhesive, coating or the like, to the annular plane-surface of the profile edge of the final-shape can, onto which the film is sealed, for example by pressure, heat, thermal radiation, ultrasound or the like, it also being possible to use a combination of these measures, as is part of the prior art. The sealed can can then be closed with a lid in a customary manner. Advantageously, an inner protective lining, composed especially of a sealable coating or the like, is applied to the inner side of the can in a manner known per se, said inner protective lining advantageously already being applied before the shaping of the can and the rolled edge. According to a preferred embodiment, a bead runs laterally around the can, which bead serves for the stiffening of said can and possibly as a stop for a lid to be fitted, as is known per se. Further beads or other beads can be provided in a manner known per se. Moreover, the base of the can according to the invention is advantageously slightly inwardly curved, but can also be planar if desired.

Aluminum or an aluminum alloy is advantageously used as material for the can according to the invention, though also suitable is sheet steel that has been surface-treated, such as tin-plated, chrome-plated, aluminized, finished, plastics-coated, etc.

The cans according to the invention can be produced in a manner known per se. In particular, the cut edge of the flange-shaped edge of the deep-drawn can body clamped on a revolving mandrel is, by means of a spinning tool designed as a profile disk and acting tangentially in relation to its central axis, pushed outward under rotation to give a profile edge, which then has on its upper side a semicircular profile, which is subsequently plastically deformed using a plane-surface flat roller with axial movement and, at the same time, flattened, giving rise to a completely planar, annular plane-surface. An advantageous embodiment of the can according to the invention and also a prior-art can are depicted in the drawing.

The cosmetic or dermatological preparations according to the invention can be put together as is customary and can serve for the treatment, the care and the cleansing of skin and/or of hair and as makeup product in decorative cosmetics. They contain preferably from 0.001% by weight to 10% by weight, preferably from 0.05% by weight to 5% by weight, especially from 0.1 to 2.0% by weight, based on the total weight of the preparations, of active-ingredient combinations used according to the invention.

It is preferred according to the invention that those active-ingredient combinations containing complexing agents be added to the active-ingredient combinations or cosmetic or dermatological preparations that are used according to the invention.

Complexing agents are excipients in cosmetology and in medical pharmaceutics that are known per se. The complexation of interfering metals such as Mn, Fe, Cu and others can, for example, prevent undesired chemical reactions in cosmetic or dermatological preparations.

Complexing agents, especially chelating agents, form complexes with metal atoms, which complexes are metallacycles in the presence of one or more polybasic complexing agents, i.e., chelating agents. Chelates are compounds in which a single ligand occupies more than one coordination site on a central atom. In this case, compounds which are normally extended are thus closed to form rings as a result of complex formation via a metal atom or metal ion. The number of bonded ligands depends on the coordination number of the central metal. A prerequisite for chelate formation is that the compound reacting with the metal contains two or more atom groups which act as electron donors.

The complexing agent(s) can advantageously be selected from the group of customary compounds, wherein preferably at least one substance from the group consisting of tartaric acid and the anions thereof, citric acid and the anions thereof, aminopolycarboxylic acids and the anions thereof (such as, for example, ethylenediaminetetraacetic acid (EDTA) and the anions thereof, nitrilotriacetic acid (NTA) and the anions thereof, hydroxyethylenediaminotriacetic acid (HOEDTA) and the anions thereof, diethyleneaminopentaacetic acid (DPTA) and the anions thereof, trans-1,2-diaminocyclohexanetetraacetic acid (CDTA) and the anions thereof).

According to the invention, the complexing agent(s) are advantageously present in cosmetic or dermatological preparations preferably to an extent of from 0.01% by weight to 10% by weight, preferably to an extent of from 0.05% by weight to 5% by weight, especially preferably to an extent of from 0.1 to 2.0% by weight, based on the total weight of the preparations.

For use, the cosmetic or dermatological preparations are, according to the invention, applied in a sufficient amount to skin and/or hair in the manner customary for cosmetics.

Cosmetic and dermatological preparations according to the invention can be present in various forms. For example, they can be a solution, an anhydrous preparation, a water-in-oil (W/O) or oil-in-water (O/W) emulsion or microemulsion, a multiple emulsions, for example a water-in-oil-in-water (W/O/W) emulsion, a gel, a solid stick, an ointment or else an aerosol. It is also advantageous to administer isoquercitrin in encapsulated form, for example encapsulated in collagen matrices and other customary encapsulation materials, for example as cellulose encapsulations, in gelatin, wax matrices or liposomally. Wax matrices in particular, as described in DE-OS 43 08 282, have been found to be favorable.

It is also possible and advantageous in the context of the present invention to include active-ingredient combinations used according to the invention in aqueous systems or surfactant preparations for the cleansing of skin and of hair.

The cosmetic and dermatological preparations according to the invention can contain cosmetic excipients, as are customarily used in such preparations, for example preservatives, bactericides, perfumes, antifoams, dyes, pigments having a coloring action, thickeners, surface-active substances, emulsifiers, emollients, moisturizers and/or humectants, fats, oils, waxes or other customary constituents of a cosmetic or dermatological formulation such as alcohols, polyols, polymers, foam stabilizers, electrolytes, organic solvents or silicone derivatives.

Emulsions according to the invention are advantageous and contain, for example, the aforementioned fats, oils, waxes and other fatty materials, and also water and an emulsifier, as is customarily used for this type of formulation.

The lipid phase can advantageously be selected from the following group of substances:
mineral oils, mineral waxes
oils, such as triglycerides of capric acid or of caprylic acid, ester oils, such as dicaprylyl ether, also natural oils such as, for example, castor oil;
fats, waxes and other natural and synthetic fatty materials, preferably esters of fatty acids with alcohols of low carbon number, for example with isopropanol, propylene glycol or glycerol, or esters of fatty alcohols with alkanoic acids of low carbon number or with fatty acids, or natural waxes, such as shea butter;
alkyl benzoates;
silicone oils such as dimethicones, dimethylpolysiloxanes, diethylpolysiloxanes, diphenylpolysiloxanes and also mixed forms thereof.

The oil phase of the emulsions, oleogels and hydrodispersions or lipodispersions within the context of the present invention is advantageously selected from the group of esters of saturated and/or unsaturated, branched and/or unbranched alkanecarboxylic acids of a chain length of from 3 to 30 carbon atoms and saturated and/or unsaturated, branched and/or unbranched alcohols of a chain length of from 3 to 30 carbon atoms, from the group of esters of aromatic carboxylic acids and saturated and/or unsaturated, branched and/or unbranched alcohols of a chain length of from 3 to 30 carbon atoms. Such ester oils can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and also synthetic, semisynthetic and natural mixtures of such esters, for example jojoba oil.

Furthermore, the oil phase can advantageously be selected from the group of branched and unbranched hydrocarbons and hydrocarbon waxes, of silicone oils, of dialkyl ethers, the group of saturated or unsaturated, branched or unbranched alcohols, and also of fatty acid triglycerides, particularly of triglycerol esters of saturated and/or unsaturated, branched and/or unbranched alkanecarboxylic acids of a chain length of from 8 to 24, especially 12-18, carbon atoms. For example, the fatty acid triglycerides can advantageously be selected from the group of synthetic, semisynthetic and natural oils, for example olive oil, sunflower oil, soybean oil, peanut oil, rapeseed oil, almond oil, palm oil, coconut oil, palm kernel oil and so on.

Any desired mixtures of such oil and wax components can advantageously be used too within the context of the present invention. It may also be advantageous to use waxes, for example cetyl palmitate, as sole lipid component of the oil phase.

Advantageously, the oil phase is selected from the group 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate and also mixtures of C₁₂₋₁₅-alkyl benzoate, 2-ethylhexyl isostearate and isotridecyl isononanoate are particularly advantageous.

The hydrocarbons which can advantageously be used within the context of the present invention are paraffin oil, squalane and squalene.

Advantageously, the oil phase can further comprise a content of cyclic or linear silicone oils or consist entirely of such oils, though preference is given to using, besides the silicone oil or silicone oils, an additional content of other oil-phase components.

Advantageously, dimethicone is used as the silicone oil to be used according to the invention. But other silicone oils too can advantageously be used within the context of the present invention, for example hexamethylcyclotrisiloxane, polydimethylsiloxane, poly(methylphenylsiloxane).

The aqueous phase of the preparations according to the invention optionally advantageously contain alcohols, diols or polyols of low carbon number, and also the ethers thereof, preferably ethanol, isopropanol, propylene glycol, glycerol, ethylhexylglycerin, the ethylene glycol, ethylene glycol monoethyl or monobutyl ether, propylene glycol monomethyl, monoethyl or monobutyl ether, diethylene glycol monomethyl or monoethyl ether and analogous products, also alcohols of low carbon number, for example ethanol, isopropanol, 1,2-propanediol, glycerol, and also especially one or more thickeners, which can advantageously be selected from the group silicon dioxide, aluminum silicates, polysaccharides and the derivatives thereof, for example hyaluronic acid, xanthan gum, hydroxypropylmethylcellulose, particularly advantageously from the group of polyacrylates, preferably a polyacrylate from the group of so-called Carbopols, for example the Carbopol types 980, 981, 1382, 2984, 5984, TR2, TR1 on their own or in combination.

In particular, mixtures of the aforementioned solvents are used. In the case of alcoholic solvents, water can be a further constituent.

Emulsions according to the invention are advantageous and contain, for example, the aforementioned fats, oils, waxes and other fatty materials, and also water and an emulsifier, as is customarily used for this type of formulation.

Gels according to the invention customarily contain alcohols of low carbon number, for example ethanol, isopropanol, 1,2-propanediol, glycerol, and water and/or an aforementioned oil in the presence of a thickener, which is preferably silicon dioxide or an aluminum silicate in the case of oily/alcoholic gels and preferably a polyacrylate in the case of aqueous/alcoholic or alcoholic gels.

Advantageously, preparations according to the invention can also contain further substances which absorb UV radiation in the UVA and/or UVB region, the total amount of the filter substances being, for example, from 0.1% by weight to 30% by weight, preferably from 0.5 to 10% by weight, especially from 1.0 to 6.0% by weight, based on the total weight of the preparations, in order to provide cosmetic preparations which protect hair and/or skin from the entire range of ultraviolet radiation.

The following examples are intended to describe the invention in more detail. Unless otherwise specified, the data refer to % by weight, based on the total weight of the composition (Z).

| Example No. | 1 | 2 | 3 | 4 | 5 |
|---|---|---|---|---|---|
| Paimitic acid + stearic | 0.9 | | | 1 | |
| acid + myristic acid + | | | | | |
| arachidonic acid + | | | | | |
| oleic acid | | | | | |
| Polyglyceryl-10 | | 1.2 | 0.8 | | 1.2 |
| stearate | | | | | |
| Glyceryl stearate | 0.85 | 1.2 | 0.8 | 0.99 | 1.2 |
| Cetylstearyl alcohol | | 3.2 | 2.4 | | 3.2 |
| Cetyl alcohol | 1 | | | 1.2 | |
| Dimethicone | 0.25 | 0.9 | 0.9 | 0.25 | 0.9 |
| C12-15 Alkyl benzoate | | 2.5 | 1.8 | | 2.2 |
| Isopropyl stearate | 2.9 | | | 3.1 | |
| Isopropyl palmitate | | 3.9 | 3 | | 3.9 |
| Carbomer | 0.7 | 0.2 | 0.2 | 0.7 | 0.2 |
| Acrylates/C10-30 alkyl | 0.04 | 0.4 | 0.2 | 0.06 | 0.1 |
| acrylate crosspolymer | | | | | |
| Xanthan gum | | 0.2 | | | |
| Glycerol | 10 | 10 | 5 | 5 | 10 |
| Phenoxyethanol | 0.25 | 0.5 | 0.5 | 0.25 | 0.5 |
| Alcohol denat. 96% | 10 | 3 | 3 | 10 | 3 |
| Ethylhexylglycerin | | 0.5 | 0.5 | | 0.5 |
| Distarch phosphate | 0.5 | 0 | 1 | 1.5 | 0 |
| Water + trisodium EDTA | | 1 | 1 | | 1 |
| Octocrylene | | 2 | 1 | | 2 |
| Butyl | | 2 | 1 | | 3 |
| methoxydibenzoylmethane | | | | | |
| Phenylbenzimidazole | | 2.25 | | | |
| sulfonic acid | | | | | |
| Water + NaOH (45%) | 0.65 | 1.15 | 0.3 | 0.85 | 0.35 |
| Water | to | to | to | to | to |
| | 100 | 100 | 100 | 100 | 100 |

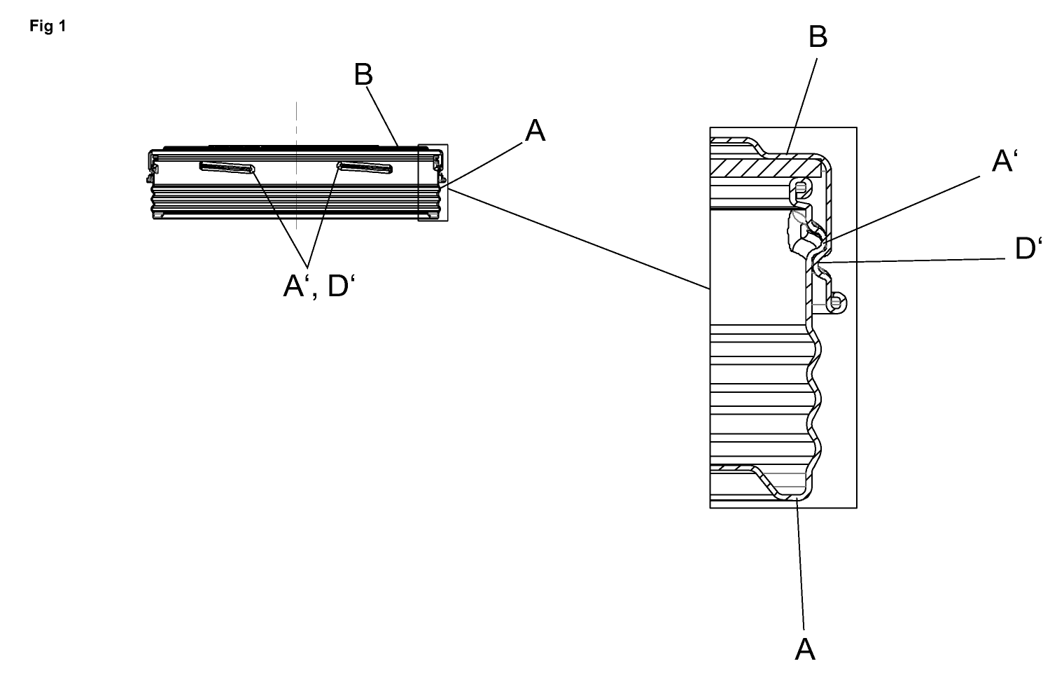

The preparations according to the examples are filled into aluminum cans according to FIGS. 1-3.

FIG. 1 shows a sectional drawing of a metal can used according to the invention, comprising
aa) a metallic container part (A) with rotation thread (A′),
   aaa) in the interior of which a cosmetic preparation (Z) is present,
   and also
bb) a lid part (D) with a thread (D′) made of the same metal as the container part,
   wherein the lid part (D) is unscrewable and screwable onto the container part (A) in a closing manner by means of the lid thread (D′) and the container thread (A′) via rotational movement, i.e., designed to be openable and reclosable via screwing movement.

Furthermore, in FIG. 1 one detail of the wall of the can and of the lid, where especially thread parts A′ and D′ are singled out.

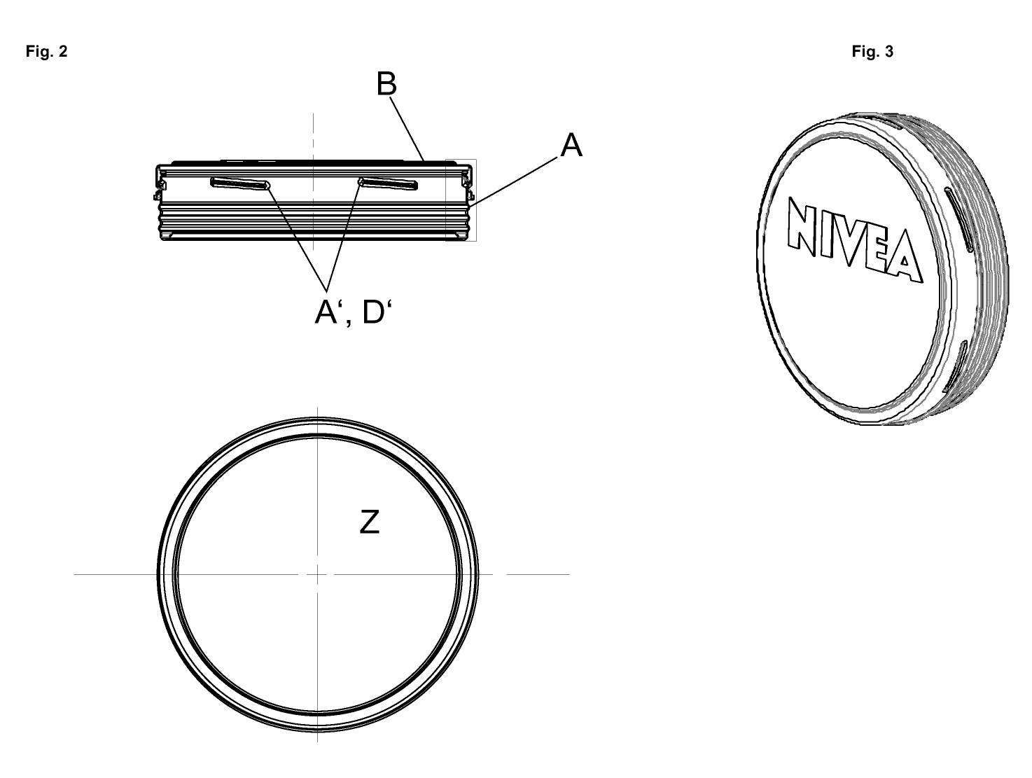

FIG. 2 shows the sectional drawing from FIG. 1 again and a horizontal cut through the complete cosmetic product, i.e., the can containing the cosmetic preparation (Z)

FIG. 3 shows a perspective drawing of the cosmetic product with commercial lettering.

1.-3. (canceled)

4. A cosmetic product, wherein the product comprises a metal can which comprises a metallic container (A) containing a cosmetic preparation (Z) and comprising a rotation thread (A′) and a lid (D) having a thread (D′) and being made of the same metal as the container (A), lid (D) being unscrewable and screwable onto container (A) in by thread (D′) and thread (A′) via rotational movement, and wherein the cosmetic preparation (Z) comprises an emulsion of one or more hydrophilic phases and one or more lipophilic phases, the one or more lipophilic phases comprising at least one lipid having a viscosity at 25° C. of less than 15 mPa·s and a spreading value at 25° C. of at least 700 mm²/10 minutes.

5. The cosmetic product of claim 4, wherein the at least one lipid comprises one or more of palmitic acid, stearic acid, myristic acid, arachidonic acid, oleic acid, glyceryl stearate, cetyl alcohol, cetearyl alcohol, C12-15 alkyl benzoate, polyglyceryl-10 stearate, isopropyl stearate, isopropyl palmitate, dimethicone.

6. The cosmetic product of claim 4, wherein the preparation comprises up to 35% by weight of the one or more lipophilic phases.

7. The cosmetic product of claim 4, wherein the preparation further comprises one or more complexing agents.

8. The cosmetic product of claim 4, wherein the preparation further comprises one or more UV filter substances.

9. The cosmetic product of claim 4, wherein the metal can consists of aluminum.

10. The cosmetic product of claim 9, wherein a protective lining is present on an inner side of the can.
